(19) United States
(12) Patent Application Publication
(10) Pub. No.: US 2025/0222150 A1
(43) Pub. Date: Jul. 10, 2025

(54) ULTRASONIC-ASSISTED HYDROGEN PEROXIDE STERILIZATION METHOD AND DEVICE

(71) Applicant: ZHEJIANG UNIVERSITY, Hangzhou (CN)

(72) Inventors: Donghong LIU, Hangzhou (CN); Ruiling LV, Hangzhou (CN); Wenjun WANG, Hangzhou (CN); Jianwei ZHOU, Hangzhou (CN); Tingting CHEN, Hangzhou (CN); Songfeng YU, Hangzhou (CN); Yi CHEN, Hangzhou (CN)

(73) Assignee: ZHEJIANG UNIVERSITY, Hangzhou (CN)

(21) Appl. No.: 18/605,676

(22) Filed: Mar. 14, 2024

(30) Foreign Application Priority Data
Jan. 5, 2024 (CN) 202410017219.6

(51) Int. Cl. A61L 2/22; A61L 2/26; B65B 55/10

(52) U.S. Cl. CPC A61L 2/22; A61L 2/26; B65B 55/10; A61L 2202/11; A61L 2202/13; A61L 2202/14; A61L 2202/15

(57) ABSTRACT

An ultrasonic-assisted hydrogen peroxide sterilization method and device are provided. The sterilization device includes a vaporization chamber, which has an inlet at an upper side and an outlet at a lower side, and a packaging film can be extended into the vaporization chamber from the inlet and extended out of the vaporization chamber from the outlet. An ultrasonic atomizer is arranged at a top of the vaporization chamber, which is externally connected to a liquid storage tank through a pipeline with a valve, liquid hydrogen peroxide is stored in the liquid storage tank, and the liquid hydrogen peroxide is atomized into hydrogen peroxide steam through an ultrasonic atomizer vibrator. A slit nozzle is arranged at an atomization outlet of the ultrasonic atomizer, through which the hydrogen peroxide steam is sprayed on a surface of the packaging film in the vaporization chamber, thus achieving rapid sterilization of the packaging film.

### CROSS-REFERENCE TO RELATED APPLICATION

This application claims foreign priority benefits under 35 U.S.C. § 119(a)-(d) to Chinese Patent Application No. 202410017219.6 filed on Jan. 5, 2024, the disclosure of which is hereby incorporated herein by reference in its entirety.

### TECHNICAL FIELD

The present disclosure relates to the technical field of sterilization, and in particular to an ultrasonic-assisted hydrogen peroxide sterilization method and device.

### BACKGROUND

Hydrogen peroxide has the chemical formula of H₂O₂, aqueous solution of which, commonly known as hydrogen peroxide solution, is a colorless and transparent liquid, and is a strong oxidant suitable for wound disinfection, environmental and food disinfection.

The bactericidal performance of liquid hydrogen peroxide has been recognized for over 100 years, which has the characteristics of environmental protection and high efficiency. However, it requires high concentration and long exposure time for the liquid hydrogen peroxide to have sporicidal ability. Later, it was found that gaseous hydrogen peroxide has a higher sporicidal ability in low concentration states than in liquid states, the main principle is that free hydrogen radicals are produced, which act on cellular components, including lipids, proteins, and DNA, so as to achieve efficient inactivation of bacteria. Therefore, there are hydrogen peroxide vaporization methods and vaporization generators in the market to vaporize the hydrogen peroxide into gas form. Hydrogen peroxide steam is obtained by heating and vaporizing the hydrogen peroxide solution. Since the unit volume is increased by 1,000 times when the liquid turns into gas, the vaporized hydrogen peroxide can quickly fill the entire sterilization space, and has stronger penetration ability, shorter sterilization period, and stronger economy than spraying.

The principles of existing hydrogen peroxide vaporization methods and devices can be roughly divided into:

(1) Heating flash evaporation method: the liquid hydrogen peroxide is dripped on a high-power heater to evaporate the liquid hydrogen peroxide into a vapor state. This method for vaporizing the hydrogen peroxide is simple, low in cost, and widely used, but has the following disadvantages: \textcircled{1} the vaporized hydrogen peroxide steam is wide in particle size distribution range, and due to the fact that the particle size of the steam cannot be controlled, the steam is easy to condense into droplets, leading to poor sterilization effect; \textcircled{2} heating power consumption is too high, leading to large sterilization energy consumption; \textcircled{3} the vaporized hydrogen peroxide is at a high temperature, which is easy to condensation when cold, and is condensed into a liquid state; and \textcircled{4} the vaporized hydrogen peroxide is wide in particle size distribution, so the vaporized hydrogen peroxide is easy to attach to a surface of objects and difficult to remove, with a large residue.

(2) High-pressure gas spraying method: based on the principle of Venturi tube, high-pressure gas is used to blow laterally a micro tube inserted into hydrogen peroxide liquid, and the liquid is charged in mist when blown out. This method is easy to achieve, low in cost, and simple. However, it has the following disadvantages: \textcircled{1} the vaporized hydrogen peroxide mist is wide in particle size distribution, which is easy to condense into droplets, leading to poor sterilization effect. Furthermore, large particles are easy to settle and attach to a surface of objects, and the residue is large and is difficult to remove; \textcircled{2} the spray system is bulky, which is not conducive to miniaturization of equipment; and \textcircled{3} the gas consumption is large, and the high-pressure harmful gas can be used only after being treated separately.

Furthermore, the existing hydrogen peroxide vaporization generator has a simple structure, and the sterilization effect of the existing hydrogen peroxide vaporization generator is limited because the vaporized particles with mist droplets are large and fewer free hydroxyl radicals are produced.

### SUMMARY

An objective of the present disclosure is to provide an ultrasonic-assisted hydrogen peroxide sterilization method and device, so as to solve the problems in the prior art. The vaporized hydrogen peroxide has a small particle size, more free hydroxyl radicals are produced, and the sterilization effect is improved.

To achieve the objective above, the present disclosure provides the following technical solution.

An ultrasonic-assisted hydrogen peroxide sterilization device includes a vaporization chamber, an inlet is formed at an upper side of the vaporization chamber and an outlet is formed at a lower side of the vaporization chamber, which is used for extending the packaging film into the vaporization chamber from the inlet and extended out of the vaporization chamber from the outlet; an ultrasonic atomizer, arranged at a top of the vaporization chamber, the ultrasonic atomizer is externally connected to a liquid storage tank and capable of atomizing liquid hydrogen peroxide into hydrogen peroxide steam by means of ultrasonic waves; and a slit nozzle, arranged at an atomization outlet of the ultrasonic atomizer, the hydrogen peroxide steam is sprayed on a surface of the packaging film in the vaporization chamber through the slit nozzle.

Optionally, the device further includes a heating portion. The heating portion is arranged at a bottom of the vaporization chamber for heating and decomposing residual hydrogen peroxide after sterilization.

Optionally, multiple rollers are sequentially arranged in the vaporization chamber at intervals in a staggered manner, and the packaging film entering the vaporization chamber from the inlet is capable of passing through the multiple rollers in sequence and exiting the vaporization chamber from the outlet.

Optionally, the ultrasonic atomizer includes an ultrasonic generator and an oscillating spring, and the liquid storage tank communicates with an interior of the oscillating spring through a pipeline.

Optionally, a peristaltic pump is arranged at the pipeline.

Optionally, the slit nozzle is arranged at the atomization outlet at the bottom of the ultrasonic atomizer.

Optionally, a concentration sensor and a temperature sensor are mounted in the vaporization chamber, which are configured to detect temperature and concentration of atomized hydrogen peroxide in the vaporization chamber, respectively.

The present disclosure further provides an ultrasonic-assisted hydrogen peroxide sterilization method, including the following steps:
Step 1: opening a liquid storage tank and turning on an ultrasonic atomizer to atomize liquid hydrogen peroxide by ultrasonic waves;
Step 2: in case that concentration of atomized hydrogen peroxide in a vaporization chamber reaches a set value, enabling a packaging film to extend into the vaporization chamber from an inlet, and sterilizing a surface of the packaging film; and
Step 3: enabling the sterilized packaging film to exit the vaporization chamber from an outlet after residual hydrogen peroxide on the surface is heated and decomposed.

Compared with the prior art, the present disclosure achieves the following technical effects:

According to the present disclosure, the hydrogen peroxide is pumped into the ultrasonic generator and the oscillating spring, and is atomized into hydrogen peroxide steam by means of ultrasonic waves. The ultrasonic waves are used to promote the atomization of the hydrogen peroxide as well as the production of free hydroxyl radicals, thus improving the sterilization efficiency of the hydrogen peroxide. The hydrogen peroxide steam is uniformly sprayed on the surface of food packaging materials through the slit nozzle to achieve the effect of rapid sterilization. Finally, the residual hydrogen peroxide is decomposed at high temperature in the vaporization chamber.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still obtain other drawings from these accompanying drawings without creative efforts.

FIG. 1 is a structural schematic diagram of an ultrasonic-assisted hydrogen peroxide sterilization device according to the present disclosure.

In the drawings: 1—ultrasonic atomizer; 2—slit nozzle; 3—packaging film; 4—inlet; 5—outlet; 6—peristaltic pump; 7—liquid storage tank; 8—vaporization chamber; 9—roller.

### DETAILED DESCRIPTION

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide an ultrasonic-assisted hydrogen peroxide sterilization method and device, so as to solve the problems in the prior art. The vaporized hydrogen peroxide has a small particle size, more free hydroxyl radicals are produced, and the sterilization effect is improved.

In order to make the objectives, technical solutions and advantages of the present disclosure more clearly, the present disclosure is further described in detail below with reference to the attached drawings and the embodiments.

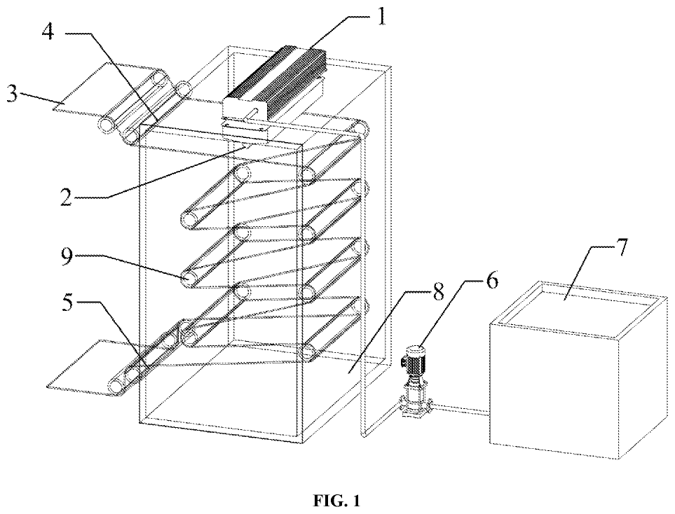

As shown in FIG. 1, an ultrasonic-assisted hydrogen peroxide sterilization device includes a vaporization chamber, an inlet 4 is formed at an upper side of the vaporization chamber and an outlet 5 is formed at a lower side of the vaporization chamber, a packaging film 3 is extended into the vaporization chamber 8 from the inlet 4 and extended out of the vaporization chamber 8 from the outlet 5. An ultrasonic atomizer 1 is arranged at a top of the vaporization chamber 8, and includes an ultrasonic generator and an oscillating spring. The ultrasonic atomizer 1 is externally connected to a liquid storage tank 7 through a pipeline with a valve, liquid hydrogen peroxide is stored in the liquid storage tank 7, and a peristaltic pump 6 is arranged at the pipeline. The liquid hydrogen peroxide can be atomized into hydrogen peroxide steam by means of of ultrasonic waves of the ultrasonic atomizer 1. A nozzle is arranged at an atomization outlet of the ultrasonic atomizer 1, and the nozzle is provided as a slit nozzle 2 which is arranged at the atomization outlet at a bottom of the ultrasonic atomizer 1, through which the hydrogen peroxide steam can be sprayed on a surface of the packaging film 3 in the vaporization chamber 8, thus achieving rapid sterilization of the packaging film 3.

In a specific embodiment, in order to avoid safety hazards caused by excess hydrogen peroxide exiting the vaporization chamber 8 with the packaging film 3 after sterilization, a heating portion is arranged at a bottom of the vaporization chamber 8 in this embodiment for heating and decomposing the residual hydrogen peroxide after sterilization.

In order to prevent the packaging film 3 from winding and knotting in the vaporization chamber 8, in this embodiment, multiple rollers 9 are sequentially arranged in the vaporization chamber 8 at intervals in a staggered manner, and an end of each roller 9 is in transmission connection with a driving motor through a chain or belt. During operation, the rollers 9 rotate, so the packaging film 3 entering the vaporization chamber 8 from the inlet passes through the rollers 9 in sequence and exit the vaporization chamber 8 from the outlet 5, and the multiple rollers 9 which are arranged at intervals in a staggered manner make the packaging film 3 in an unfold state, so the contact surface area of the packaging film 3 with the hydrogen peroxide is larger, and the sterilization effect is better.

In order to monitor temperature and concentration of the atomized hydrogen peroxide in the vaporization chamber 8 in real time, a concentration sensor and a temperature sensor are mounted in the vaporization chamber 8 to detect the temperature and the concentration of the atomized hydrogen peroxide in the vaporization chamber 8, respectively.

The present disclosure further provides an ultrasonic-assisted hydrogen peroxide sterilization method, the ultrasonic waves are used to promote the atomization of the hydrogen peroxide as well as the production of free hydroxyl radicals, thus improving the sterilization efficiency of the hydrogen peroxide. The method includes the following steps:

Step 1. A liquid storage tank 7 is opened, and the ultrasonic atomizer 1 is turned on to atomize liquid hydrogen peroxide by ultrasonic waves. After the hydrogen peroxide passes through the nozzle of the ultrasonic oscillating spring, the hydrogen peroxide is vaporized into hydrogen peroxide steam by means of oscillation of the ultrasonic waves. Meanwhile, in the process of vaporization, the ultrasonic waves also promote the reaction (1) and the formation of hydroxyl radicals, thus obviously enhancing the sterilization effect of the hydrogen peroxide steam.

H₂O→H·+·OH (1);

Step 2. In case that concentration of atomized hydrogen peroxide in the vaporization chamber 8 reaches a set value, a packaging film 3 is extended into the vaporization chamber 8 from the inlet, and a surface of the packaging film 3 is sterilized.

Step 3. After the residual hydrogen peroxide on the surface is heated and decomposed, the sterilized packaging film 3 is enabled to exit the vaporization chamber 8 from an outlet 5 to be far away from the vaporization chamber 8.

The above solution is described with one specific embodiment.

Embodiment 1:1 mM of hydrogen peroxide solution is treated for 60 min by ultrasonic waves of 200 W and 20 KHz.

Comparative example 1-1: The ultrasonic duration in Embodiment 1 is changed to 10 min, and the rest is same as that in Embodiment 1.

Comparative example 1-2: The ultrasonic duration in Embodiment 1 is changed to 30 min, and the rest is same as that in Embodiment 1.

Embodiment 2: Hydrogen peroxide solution with concentration of 30% is atomized by an ultrasonic atomization device at spraying speed of 0.2 mL/min, and diameter of the atomized hydrogen peroxide is 5 μm. Movement speed of the packaging film is 20 m/min, and temperature of the vaporization chamber is 120° C. E. coli suspension of 2.0×108 CFU/mL is evenly applied on the packaging film, and the packaging film passes through the slit nozzle and vaporization chamber at speed of 20 m/min.

Comparative example 2-1: The concentration of hydrogen peroxide in Embodiment 2 is changed to 10%, and the rest is same as that in Embodiment 1.

Comparative example 2-2: The ultrasonic treatment in Embodiment 2 is canceled, the hydrogen peroxide is vaporized at instantaneous high temperature, and the rest is same as that in Embodiment 2.

Experiment 1 Concentration of Hydroxyl Radicals in Hydrogen Peroxide Solution after Ultrasonic Treatment:

Terephthalic acid (TA) can be converted into 2-hydroxyterephthalic acid (HTA) by the hydroxyl radicals. The HTA has fluorescence characteristics and an emission wavelength of 425 nm at an excitation wavelength of 310 nm.

TABLE 1
Concentration of hydroxyl radicals in hydrogen peroxide
solution after different ultrasonic treatments Unit: μM

| | Concentration of hydroxyl radicals |
|---|---|
| Different treatments | (μM) |
| Blank control | 0.02 ± 0.01 |
| Embodiment 1 | 19.06 ± 1.62 |
| Comparative example 1-1 | 3.82 ± 0.36 |
| Comparative example 1-2 | 10.09 ± 1.25 |

Cavitation bubbles are produced when the ultrasonic waves propagate in water, and the cavitation bubbles grow and collapse as the sound pressure increases continuously, resulting in instantaneous high temperature, high pressure, and producing micro-jets and free radicals. As can be seen from the data in Table 1, the ultrasonic treatment can promote the production of hydroxyl radicals in the hydrogen peroxide solution, and the hydroxyl radicals can be increased with the increase of ultrasonic treatment time.

### Experiment 2: Verification of Sterilization Effect of Hydrogen Peroxide Steam in Different States

10 cm² of the packaging film obtained from each of Embodiment 2 and all Comparative examples and the blank control (the packaging film inoculated with E. coli suspension without treatment is used as blank control) are rinsed repeatedly with 10 mL of sterile physiological saline, and evenly coated with 20 mL of nutrient agar culture medium. The number of bacteria is counted after the packaging films are cultured for 24 h at 37° C. to compare the inactivation efficiency of Escherichia coli by different treatment methods.

TABLE 2
Number of survival bacterial colonies after hydrogen peroxide
treatment in different states Unit: log(CFU/cm²)

| Different treatments | Number of bacterial colonies |
|---|---|
| Blank control | 7.21 ± 0.04 |
| Embodiment 2 | 0.76 ± 0.05 |
| Comparative example 2-1 | 4.18 ± 0.00 |
| Comparative example 2-2 | 3.01 ± 0.02 |

Compared with the existing high-temperature vaporizing hydrogen peroxide, the ultrasonic waves can promote the production of hydroxyl radicals while vaporizing the hydrogen peroxide into steam, such that the sterilization ability of the hydrogen peroxide is improved, and the packaging film can be rapidly sterilized in the rapid movement process.

In the description of the present disclosure, it needs to be understood that the orientation or positional relationship indicated by terms “center”, “top”, “bottom”, “left”, “right”, “vertical”, “horizontal”, “interior” and “outside” is based on the orientation or positional relationship shown in the drawings only for convenience of description of the present disclosure and simplification of description rather than indicating or implying that the device or element referred to must have a particular orientation, be constructed and operate in a particular orientation, and thus are not to be construed as limiting the present disclosure. Furthermore, the terms “first” and “second” are used for descriptive purposes only and are not to be construed as indicating or implying relative importance.

Specific examples are used herein for illustration of the principles and implementation methods of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. An ultrasonic-assisted hydrogen peroxide sterilization device, comprising:
a vaporization chamber, an inlet is formed at an upper side of the vaporization chamber and an outlet is formed at a lower side of the vaporization chamber, a packaging film is extended into the vaporization chamber from the inlet and extended out of the vaporization chamber from the outlet;
an ultrasonic atomizer, arranged at a top of the vaporization chamber, the ultrasonic atomizer is externally connected to a liquid storage tank and capable of atomizing liquid hydrogen peroxide into hydrogen peroxide steam by means of ultrasonic waves; and
a slit nozzle, arranged at an atomization outlet of the ultrasonic atomizer, the hydrogen peroxide steam is sprayed on a surface of the packaging film in the vaporization chamber through the slit nozzle.

2. The ultrasonic-assisted hydrogen peroxide sterilization device according to claim 1, further comprising a heating portion, the heating portion is arranged at a bottom of the vaporization chamber for heating and decomposing residual hydrogen peroxide after sterilization.

3. The ultrasonic-assisted hydrogen peroxide sterilization device according to claim 1, wherein a plurality of rollers are sequentially arranged in the vaporization chamber at intervals in a staggered manner, and the packaging film entering the vaporization chamber from the inlet passes through the plurality of rollers in sequence and exits the vaporization chamber from the outlet after.

4. The ultrasonic-assisted hydrogen peroxide sterilization device according to claim 1, wherein the ultrasonic atomizer comprises an ultrasonic generator and an oscillating spring, and the liquid storage tank communicates with an interior of the oscillating spring through a pipeline.

5. The ultrasonic-assisted hydrogen peroxide sterilization device according to claim 4, wherein a peristaltic pump is arranged at the pipeline.

6. The ultrasonic-assisted hydrogen peroxide sterilization device according to claim 1, wherein the slit nozzle is arranged at the atomization outlet at a bottom of the ultrasonic atomizer.

7. The ultrasonic-assisted hydrogen peroxide sterilization device according to claim 1, wherein a concentration sensor and a temperature sensor are mounted in the vaporization chamber to detect temperature and concentration of atomized hydrogen peroxide in the vaporization chamber, respectively.

8. An ultrasonic-assisted hydrogen peroxide sterilization method, comprising the following steps:
Step 1: opening a liquid storage tank and turning on an ultrasonic atomizer to atomize liquid hydrogen peroxide by means of ultrasonic waves;
Step 2: when concentration of atomized hydrogen peroxide in a vaporization chamber reaches a set value, enabling a packaging film to extend into the vaporization chamber from an inlet, and sterilizing a surface of the packaging film; and
Step 3: enabling the sterilized packaging film to exit the vaporization chamber from an outlet after residual hydrogen peroxide on the surface is heated and decomposed.
